SEQUENCE LISTING (4 sequences)

SEQ ID NO: 1 - protein, 107 aa, anti-proteases SLPI
SGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 2 - protein, 132 aa, native immature form of anti-proteases SLPI
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 3 - protein, 107 aa, oxidation-resistant mature form of anti-proteases SLPI
SGKSFKAGVCPPKKSAQCLRYKKPECQSDWQCPGKKRCCPDTCGIKCLDPVDTPNPTRRKPGKCPVTYGQCLLLNPPNFCEMDGQCKRDLKCCMGMCGKSCVSPVKA

SEQ ID NO: 4 - protein, 132 aa, oxidation-resistant immature form of anti-proteases SLPI
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLLLNPPNFCEMDGQCKRDLKCCMGMCGKSCVSPVKA

(19) United States
(12) Patent Application Publication
(10) Pub. No.: US 2009/0069257 A1
(43) Pub. Date: Mar. 12, 2009

(54) Method of achieving persistent Transgene expression

(76) Inventors: David Ennist, Bethesda, MD (US); Patrick Lu, Rockville, MD (US); Mervat Mina, Germantown, MD (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/732,158

(22) Filed: Apr. 2, 2007

Related U.S. Application Data
(63) Continuation of application No. 10/169,323, filed on Oct. 23, 2002, now abandoned.

(51) Int. Cl. A61K 31/711; C12N 15/64; A61P 25/00

(52) U.S. Cl. 514/44; 435/320.1

(57) ABSTRACT

Non-inflammatory vector compositions are provided that are suitable for repeated transgene delivery and that result in persistent transgene expression. The compositions are non-inflammatory, the present compositions are suitable for readministration and do not induce expression-limiting immune or inflammatory responses. Thus, these compositions are useful in methods of repeated administration to achieve persistent transgene expression, and are especially suited to treating genetic, acquired and inflammation-associated conditions.

The invention relates to non-inflammatory vector composition, as well as method of treating a patient suffering from a disorder having an inflammatory component

### BACKGROUND OF THE INVENTION

The lung is an attractive target for gene therapy methodologies due to its accessibility and large surface area. Existing techniques, however, suffer from certain failings. For example, ex vivo gene therapy for pulmonary diseases involving implantation into the tracheal epithelium has been studied experimentally, but faces challenges in transitioning to the clinic. For full effectiveness, this type of therapy requires the permanent genetic modification of a stable, self-renewing cell population capable of giving rise to the other cell types of the epithelium. The existence of such a stem cell for the pulmonary epithelium, however, remains controversial to this day.

An alternative ex vivo approach involves transplantation of gene-modified cells at a site distal to the lung to obtain serum secretion of a protein that is therapeutically relevant to pulmonary disease. Several studies have been reported using cells transplanted in the liver or the peritoneum bearing a transgene encoding α₁-antitrypsin, a deficiency of which causes familial emphysema. Kay, et al., Proc. Natl. Acad. Sci. USA 89: 89-93 (1992); Garver, et al., Science 237: 762-64 (1987). Low-level transient expression was observed. However, α₁-antitrypsin must reach a concentration of greater than 1 mg/ml in the serum in order to have a therapeutic effect by diffusion into the lung. This level of expression is beyond the capabilities of the best vectors currently available.

In addition, ex vivo approaches require use of syngeneic cells in order to avoid immunological rejection of the transplant. Accordingly, such methods are highly individualized and require a significant amount of tissue culture per patient. The ex vivo approach is thus both labor intensive and technically very demanding.

In contrast, in vivo gene therapy products for the lung that can be directly administered to the patient are more readily incorporated into the current medical and pharmaceutical infrastructure. For example, most cystic, fibrosis gene therapy products under development are designed for delivery by aerosol based systems similar to those currently in clinical use for the administration of a number of conventional pulmonary medicines. Martin, et al., Hum. Gene Ther. 9: 87-114 (1998). Thus, many of the technical, medical and commercial underpinnings that must be developed for the successful introduction of ex vivo gene therapies already are in place for in vivo gene therapies. Historically, in vivo systems have relied on viral delivery systems, but there is substantial interest and effort directed toward developing synthetic vector compositions, due to shortcomings associated with viral vectors.

At first blush, adenoviruses seem a good choice for a pulmonary delivery system. They are trophic for the respiratory epithelium and have a relatively large coding capacity. In 1992, the NIH Recombinant DNA Advisory Committee (RAC) approved the first three clinical gene therapy protocols for cystic fibrosis, all of which were adenoviral based. The initial enthusiasm over adenoviral vectors, however, has since been tempered by the realization that host responses severely limit the utility of this vector system. In particular, immediate inflammatory responses limit initial transduction efficiencies, alveolar macrophages rapidly eliminate adenoviral vectors, cytotoxic T lymphocyte (CTL) responses limit persistence of expression, and the ability to readminister the vector is prevented by an antibody response.

Another commonly used vehicle is adeno-associated virus (AAV), a small single stranded DNA parvovirus that requires coinfection with adenovirus or a herpesvirus for propagation. Berns, in VIROLOGY (Fields, B. N. et al., eds), pp. 1743-63 (1990). The virus is able to integrate into the genome of human cells at a unique site on chromosome 19, but the location and extent to which vectors derived from AAV integrate remains problematic. Expression of human CFTR six months following AAV-mediated gene transfer has been detected in rabbits (Flotte et al., Proc. Natl. Acad. Sci. USA 90, 10613-17 (1993)), and human clinical trials have been approved using this vector. Flotte et al., Hum. Gene Ther. 7: 1145-1159 (1996). A phase I study of an adeno-associated virus-CFTR gene vector in adult CF patients with mild lung disease (Wagner et al., Hum. Gene Ther. 9: 889-909 (1998)), and an initial report of a clinical trial have been published. Wagner et al., Lancet 351, 1702-03 (1998); Wagner et al., supra). Nevertheless, as with adenoviral vectors, preexisting antibodies may limit the usefulness of this vector, and the induction of an antibody response may prevent readministration of AAV vectors as well. Zeitlin in GENE THERAPY FOR DISEASES OF THE LUNG (Brigham, ed), pp. 53-81 (1997). Moreover, the viral coding sequences are generally provided in trans from a helper plasmid, and the plasmids are cotransfected into adenovirus infected cells to produce quantities of the AAV vector. These procedures have proven to be cumbersome and subject to contamination with adenovirus.

Lentiviruses are positive-strand RNA viruses that utilize reverse transcriptase to convert their genome into a double-stranded DNA provirus that inserts into the genome of the infected cell. Narayan et al., in VIROLOGY (Fields et al., eds), pp. 1679-1721 (1990). Unlike traditional retroviral vectors, (Miller et al., Mol. Cell. Biol. 10: 4239-42 (1990)), lentiviral vectors are able to infect nondividing cells, (Naldini, et al., Science 272, 263-67 (1996); Miyake et al., Hum. Gene Ther. 9: 467-75 (1998)), thus opening up the possibility that these vectors could be used for in vivo applications. Initial preclinical studies using an HIV based vector expressing the CFTR gene have been reported. Goldman et al., Hum. Gene Ther. 8: 2261-68 (1997). As with retroviral vectors in general, safety considerations of an integrating lentiviral vector are a substantial concern. Temin, Hum. Gene Ther. 1: 111-23 (1990).

Synthetic vectors employ a complex of nucleic acid, usually in the form of a plasmid, with molecules that facilitate the delivery of the nucleic acid to target cells. As alternatives to viral vectors, synthetic vectors should have lower toxicity and lower immunogenicity. Synthetic vectors have several potential advantages: They do not have an upper size limit to the DNA that can be packaged, are not infectious, are easier to manufacture and QC in large quantities, and are made up of well-defined components. Thus, the primary rationale for pursuing synthetic vector systems is to avoid the known problems of viral systems. Two main classes of synthetic vectors have proven to be useful in delivering genes in vivo, where DNA is complexed with cationic lipids or cationic polymers, respectively.

Plasmid DNA complexed with cationic lipids has been successfully used to transfect lung cells in vivo by both intravenous and tracheal administration. Cationic polymers similarly interact with DNA by electrostatic interactions. Polylysine has been conjugated to ligands that allow receptor-mediated delivery of the complex to specific target cells. Polyethyleneimine (PEI) is a cationic polymer that has shown some recent promise. This cationic polymer has been found to be an efficient vector for in vivo transduction of mouse lungs, either by the oral-tracheal or intravenous routes. Ferrari et al., Gene. Ther. 4, 1100-1106 (1997); Goula et al., Gene Ther. 5, 1291-1295 (1998).

The majority of in vivo animal studies using synthetic vectors have relied on DNA plasmids complexed with a cationic component to deliver the transgene. See for example, Felgner et al., Proc. Natl. Acad. Sci. 84: 7413-17 (1987); Gao in GENE THERAPY FOR DISEASES OF THE LUNG (Brigham, ed.), pp. 99-112 (1997). In comparison to viral vectors, these studies with plasmids have demonstrated uniformly low levels of transgene expression in experimental animals. On the other hand, the use of viral vectors appears to be limited by host response issues that may be minimal for synthetic vector formulations. Thus, neither system in its present conception has demonstrated a clear advantage, and both have substantial problems. It is, therefore, a goal of the present invention to overcome these difficulties in the art and to provide methods and compositions for inducing persistent, effective amounts of pulmonary and intra-articular transgene expression.

### SUMMARY OF THE INVENTION

It is an object of the invention, therefore, to provide a method of pulmonary transgene delivery that overcomes the above-identified and other deficiencies in the art. According to this object of the invention, the invention provides a composition containing a free DNA vector encoding a transgene, an enhancing agent (like a surfactant) and/or an antiinflammatory agent, (like a steroid). This composition, when administered to a patient, is capable of eliciting significant levels of transgene expression, without a limiting immune response, which makes them particularly suitable for treating inflammatory disorders. In one embodiment, the composition is administered repeatedly, typically at intervals exceeding forty-eight hours. In certain embodiments the transgene is a protease inhibitor, which can act on a variety of proteases, including neutrophil elastase, cathepsin G, collagenase, proteinase 3, plasminogen activator. The invention provides compositions and methods suitable to obtain therapeutically effective levels of transgene-encoded products sufficient to treat patients suffering from pulmonary disorders such as emphysema, chronic obstructive pulmonary disease, cystic fibrosis, adult respiratory distress syndrome and asthma, and other disorders with an inflammatory component, like arthritis.

### BRIEF DESCRIPTION OF THE DRAWINGS

FIG. 1 demonstrates sustained high levels of transgene expression using the inventive compositions and methods.

FIG. 2 demonstrates in vivo distribution of a secreted protein following oral-tracheal instillation of a plasmid carrying the cDNA of the secreted protein.

### DETAILED DESCRIPTION OF THE INVENTION

The invention is directed to methods and compositions for achieving persistent expression of a therapeutic gene product. The therapeutic gene product typically is a protein, but may also be a nucleic acid such as an antisense or ribozyme agent. Surprisingly, the inventors have found that the compositions and methods described herein do not elicit a limiting inflammatory or immune response, and this property allows for repeated nucleic acid administration without significantly reduced expression levels. More specifically, the invention provides new and improved methods of delivering a nucleic acid to the lungs or joints of an animal. In one embodiment the nucleic acid encodes a gene whose expression is controlled by other elements contained within the same vector that contains the nucleic acid. These new and improved methods and compositions are useful in both therapeutic and experimental contexts.

The present methods and compositions are useful in any situation, including clinical situations, where persistent transgene expression is desired. They are especially suited to situations requiring persistent expression in the lungs or joints. For example, they are useful in human gene therapy applications. It will be understood, however, that they may also be used in veterinary (non-human animal) applications, especially for mammals. They are also useful in generating experimental animal models of transgenes.

### 1. Compositions Useful in the Invention

The invention provides a composition suitable for delivering a transgene. Generally, such compositions are composed of a nucleic acid such as DNA, encoding at least one gene of interest (“transgene”). Some preferred compositions also contain a enhancing agent, such as a surfactant, and/or a steroid. Importantly, the vector portion of the composition does not significantly exacerbate an inflammatory condition in the lung or joints. In other words, the vector does not induce a substantial inflammatory response, which would be counter to treating inflammatory disorders. Characteristics of the inflammatory response are provided below. One preferred form of such a composition contains a nucleic acid lacking CpG islands that activate or enhance activation of lymphoid cells. These compositions may be used, for example, in the treatment of respiratory disease, particularly asthma and chronic obstructive pulmonary disease (COPD) or in the treatment of arthritic disease, such as rheumatoid arthritis and osteoarthritis.

The DNA used in the present compositions can be plasmid-based. Such vectors are conveniently propagated in bacteria, like E. Coli. However, when bacterial propagation is used, it is preferable to use purification methodologies that result in efficient endotoxin removal, since endotoxin may interfere with persistent transgene expression, likely by promoting an inflammatory response. While the vector is preferably supercoiled, for maximum transgene expression it may also be relaxed or even linearized. The artisan is well apprised of methods for generating and propagating such vectors.

In general, the vectors used in the invention contain the features that are characteristic of plasmid vectors. These include, for example, an origin of replication suitable for propagation in a host. A standard example is the ColE1 origin, which can exist as the relatively low copy number version present in pBR322 or the higher copy number version present in the pUC series of vectors and other conventional vectors. The vectors also generally contain a selectable marker to ensure that a transformed host cell retains the vector. Common examples may confer ampicillin resistance, puromycin resistance, tetracycline resistance, kanamycin resistance, rifampicin resistance or spectinomycin resistance. The skilled artisan will be aware that other selectable markers, including markers that will be developed in the future, can be used. A multiple cloning site is also beneficially included.

The vectors contain all of the cis elements needed for effective transcription and translation of the encoded transgene. These elements will be operatively linked to the transgene so as to facilitate transgene expression. Such elements, such as promoters, are well known to the artisan. Exemplary promoters may be strong constitutive promoters, may be tissue-specific, inducible or have any other known desirable characteristic.

As used herein, “free nucleic acid” is defined as an aqueous solution of nucleic acid, typically DNA, RNA, or synthetic analogues. Nucleic acids are typically prepared by biological propagation of constructs that are isolated, purified, and in some cases modified synthetically from a plasmid or viral source. Conventional viral vectors, such as recombinant retroviruses, lentiviruses, adenoviruses and adeno-associated viruses, are not included within this definition. Importantly, free nucleic acids do not induce an expression-limiting immune response; and do not exacerbate, for example, a pulmonary or intra-articular inflammatory condition. The free nucleic may be an RNA, but DNA molecules are preferred due to their superior stability.

A particular embodiment of the invention provides methods of using forms of nucleic acids that produce therapeutic activity through the expression of antisense or ribozyme constructs. In yet another embodiment of the invention, forms of nucleic acids are used that produce therapeutic activity without requiring expression by the cells of the lung or the joint. For instance, the nucleic acids themselves may be active directly, as is the case with antisense and catalytic nucleic acid molecules.

The compositions may contain an “enhancing agent” that improves the pharmacology of the nucleic acid composition within the tissues of the lung or joint, (e.g. distribution and persistence), resulting in improved therapeutic activity of the nucleic acid. One preferred enhancer is a natural or biological surfactant. Surfactants are surface-active amphipathic compositions that have surface tension-lowering properties The surfactants useful in the invention are safe for pulmonary administration. Such surfactants, both synthetic and natural, are well known in the art and several are commercially available. Examples include Survanta (Beractant, available from Ross Laboratories), Exosurf (colfosceril palmitate, available from Glaxo Wellcome), Infasurf (calfactant, available from Forest Laboratories) and other surfactants which lower the surface tension, thus facilitating the dispersion of the vector. Naturally occurring surfactants are a complex mixture of phospholipids, neutral lipids, fatty acids and proteins. Surfactants are amphipathic in nature, having polar as well as nonpolar components and, thus, permit interactions between aqueous and lipidic fluids.

Survanta, a preferred surfactant, is a semi-synthetic surfactant derived from cow lung. It contains naturally-occurring lipids, fatty acids, and the surfactant-associated proteins SP-B and SP-C. This mixture is supplemented with additional fatty acids to provide a standardized preparation. Survanta is approved for clinical use in treating infant respiratory distress syndrome (RDS). A typical dose is 4 cc/kg intratracheally and up to 4 doses, given 6-12 hours apart, are used. Similar dosing regimens are suitable for use in the present invention, though methods of determining alternative dosing regimens are known to those of skill in the art.

Some enhancing agents are synthetic or semi-synthetic surfactants. Such enhancers can be amphipathic synthetic or semi-synthetic polymers, lipids, and fluorocarbons. A suitable class of synthetic polymer surfactant is Pluronic™ surfactants. One synthetic lipid surfactant is an anionic liposome formulation (Bangham, et al., Chem-Phys-Lipids. 64: 275-85 (1993), Bangham, et al., Lung. 165: 17-25, (1987)). Other lipid surfactants are surfactant polymer-lipid conjugates. Suitable such conjugates include Thesit™, Brij 58™, Brij 78™, Tween 80™, and Chol-PEG 900. The skilled artisan will recognize that other synthetic and semi-synthetic surfactants may be used without departing from the spirit of the invention.

Other enhancing agents are particularly useful in delivering nucleic acids to the joint. In a manner analogous to the foregoing surfactants, certain compounds may be used to increase the distribution profile and other therapeutically useful characteristics of the present vectors in the joint. These compounds typically are polysaccharides, composed of linear repeating disaccharide, units. Hyaluronic acid is exemplary; it consists of disaccharide units of 1,4-linked β-D-glucuronic acid and 1,3-linked 2-acetamido-2-deoxy-β-D-glucopyranose.

Hyalgan, a preferred form of hyaluronic acid, is a viscous solution of a high molecular weight fraction of purified natural sodium hyaluronate in buffered physiological saline. It is approved for clinical use for the treatment of pain in osteoarthritis of the knee. A typical dose is 20 mg administered by intra-articular injection once a week for a total of five injections. Similar dosing regimens are suitable for use in the present invention, though methods of determining alternative dosing regimens are known to those of skill in the art.

Still other enhancing agents permit improved contact with target cells in deep airways and in structural regions or throughout the joint capsule once the nucleic acid has reached the tissue. Such enhancers can be electrically neutral, amphipathic polymers. One suitable electrically neutral, amphipathic polymer is polyoxazoline.

Yet other enhancing agents permits persistence of the nucleic acid extracellularly in the deep airways and within the joint capsule and in structural regions thereby enhancing persistence of therapeutic activity. Such enhancers reduce metabolic processes and clearance of the nucleic acid from the tissues of the lung or joint.

Additional preferred compositions contain an antiinflammatory agent, such as a steroid, that can effectively suppress or alleviate one or more aspects of an inflammatory response, including mononuclear cell infiltration, edema, release of chemokines and other pro-inflammatory mediators. Inhaled steroids for the lung or intra-articular steroid injections are particularly preferred in this regard. Examples include beclomethasone (e.g., VANCERIL, BECLOVENT—conventional dose about 42-84 mcg/Inh), triamcinolone (e.g., AZMACORT—conventional dose about 100 mcg/Inh), flunisolide (e.g., AEROBID-M, NASALIDE, BRONALIDE, RHINALAR—conventional dose 42-250 mcg/Inh), fluticasone (e.g., FLOVENT, conventional dose 50-250 mcg/Inh), budesonide (e.g., RHINOCORT—conventional dose 100-200 mcg/Inh), dexamethasone and hydrocortisone. In another embodiment of the invention, the anti-inflammatory agents can be administered systemically, for example via oral administration or intravenous administration. Examples include dexamethasone and hydrocortisone.

The antiinflammatory compound may also be a non-steroidal anti-inflammatory agent (an NSAID). Examples of suitable NSAIDs include COX2 inhibitors (e.g., CELEBREX, conventional dose 200 mg/day) and Tilade. Useful dosages are based on those conventionally used in the art. Frequency of administration also is informed by the art, but will generally be guided by how often the nucleic acid vector is administered.

It is understood that any compounds described herein contemplate, where applicable, any free acids, free bases, esters, as well as pharmaceutically acceptable salts thereof. Reference to one form, should be read as contemplating all forms, unless otherwise noted.

### 2. Methods of the Invention

A typical method entails administering to a patient an effective amount of a composition comprising a free nucleic acid vector, such as a DNA plasmid, in combination with an enhancer and/or an antiinflammatory compound. The vector encodes a transgene of interest, and the methods result in effective transgene expression. In the context of a therapeutic method, an effective amount of expression is one that is therapeutically significant, meaning that there is some measurable effect on the disease itself, symptoms and/or some underlying pathological marker. Such effects may be qualitative or quantitative, and the clinician will be familiar with each marker as it relates to different conditions being treated. For example, decreased IL8 or neutrophils in the bronchoalveolar fluid of COPD patients would be indicative of a therapeutic effect.

Surfactants and other enhancing agents useful in the inventive methods may be obtained commercially. Examples of suitable surfactants include, but are not limited to, Survanta (Beractant, available from Ross Laboratories), Pluronic™, Tween™, and Brij™. As noted, a key feature of the present methods is that no expression-limiting inflammatory response is induced, which allows for activity of the nucleic acid or persistent transgene expression. Survanta has been shown to suppress mitogen-induced lymphocyte proliferation (Kremlev et al., Am. J. Physiol. 267:L357-64 (1994)), and may thus aid in this process. Other surfactants may have similar beneficial properties.

The nucleic acid-containing composition may be delivered, for example, as a dry powder or as a liquid suspension by any suitable means that results in pulmonary administration. For example, the composition may be inhaled (e.g., as an aerosol), instilled in the lung and/or administered tracheally. The skilled artisan will recognize that these methods of administration may be used independently or in combination, as particular circumstances require. In addition, other methods of pulmonary administration may be used, including methods that are developed in the future.

Similarly, the transgene-containing composition may be delivered as a liquid suspension, or other physiologically compatible form, by any suitable means that results in intra-articular administration. For example, the composition may be directly injected into the joint or injected intravenously and targeted to the inflamed joint. The skilled artisan will recognize that these methods of administration may be used independently or in combination, as particular circumstances require. In addition, other methods of articular administration may be used, including methods that are developed in the future.

A key feature of the present methods is persistent expression of the nucleic acid activity, e.g. transgene expression. Heretofore, gene therapy methods have been limited by immune and inflammatory responses, which (1) reduced overall nucleic acid activity, in this case transgene expression and (2) prevented the use of re-administration as a method of boosting expression. Thus, while reasonable levels of transgene expression were obtained initially, the expression did not persist. Expression is “persistent” where it does not decrease substantially over time, either as a result of a single dose or multiple administrations. Usually, therefore, effective levels of expression are maintained. Preferably, expression levels do not decrease to less than 25% of the maximal level in between administrations, and more preferably not less than about 50%, but in some instances it is beneficial to go below these thresholds, such that the a dose “pulsing” is accomplished with repeated administration.

A preferred method involves administering the free nucleic acid vector compositions on two or more occasions. In this manner, nucleic acid activity may be boosted or restored to levels approximating the level obtained following the initial administration so that effective levels of nucleic acid activity are maintained. Because the present methods do not trigger a limiting immune or inflammatory response, the compositions of the invention may be administered as many times as needed to maintain effective levels of activity. In some methods, administration is accomplished every few days, but more typically it is done weekly, and some may involve biweekly or monthly administration. Using such methods, a decrease in activity between administrations may be observed, depending on the condition being treated. These methods are still considered to induce “persistent” activity because, unlike prior art methods, the present compositions can be re-administered repeatedly, without a substantial decrease in transgene expression, relative to the previous dose. Typical methods entail repeated administration for at least about a month, but may entail longer periods of treatment.

In another embodiment, the inventive method further entails administering an antiinflammatory compound, such as a steroid or an NSAID, that has the ability to mitigate a subject's inflammatory response. In general, it is useful to administer the antiinflammatory compound prior to administering the vector/enhancer composition, but they may be administered together. Suitable antiinflammatory compounds are described above. The antiinflammatory agent typically is administered by intravenous or oral route, and may be included in the nucleic acid composition, or used as a separate drug.

### 3. Therapeutic Indications

The therapeutic application of the present methods extends to pulmonary and non-pulmonary disorders, including rheumatoid arthritis and osteoarthritis. As indicated above, the lungs are an attractive target for nucleic acid delivery due to their large surface area and relative ease of access. Thus, the present methods are adaptable to the gene therapy-based treatment of any (pulmonary or non-pulmonary) disorder where therapeutic levels of transgene expression are obtained. The artisan is well versed in such applications. For example, Brigham et al., Nature 362: 250-55 (1993), expressed human growth hormone (hGH) following administration of a plasmid encoding the cDNA and Cannizzo et al., Nature Biotech. 15: 570-73 (1997), increased blood platelet counts following instillation of an adenoviral vector expressing thrombopoietin in the lungs of experimental animals.

Due to their adaptability to gene therapy, the present methods are especially suited to treat disorders of the lung and conditions that have pathological manifestations in the lung. In particular, the methods are suited for treatment of diseases with inflammatory pathologies and etiologies. As discussed in detail below, exemplary disorders include, but are not limited to, emphysema, COPD, cystic fibrosis (CF), adult respiratory distress syndrome (ARDS), pulmonary fibrotic syndromes and asthma.

Examples of genetic disorders with pulmonary manifestations suitable for the inventive treatment methods include CF and familial emphysema. CF results from a deficiency in the cystic fibrosis transmembrane regulator (CFTR), a cAMP-activated chloride channel. The disease is characterized by viscous airway secretions, chronic respiratory infections, bronchiectasis, pancreatic fibrosis, and bowel dysfunction. The respiratory manifestations predominate and death results from progressive respiratory failure in greater than 95% of cases.

Numerous clinical trials for CF gene therapy have been initiated and reports have been published using adenoviral and synthetic vectors. See, for example, (Crystal et al., Nature Genet. 8: 42-51 (1994) and Caplen et al., Nature Med. 1: 39-46 (1995). Though none of these trials have demonstrated patient benefit, they have provided evidence for gene delivery to the airway epithelium. Middleton et al., Thorax 53: 197-199 (1998); Alton et al., Gene Ther. 5: 291-92 (1998). While the levels of gene transfer and expression and the degree of electrophysiologic correction (where measured) have been uniformly low, the transgene, its mRNA and the protein product have all reached detectable levels in biopsies of treated patients. Only very low levels of CTFR need to be expressed to treat CF, and a low-level constitutive promoter may be sufficient to express levels sufficient for therapeutic benefit. Of greater importance in treating CF is a need to achieve fairly uniform transduction of cells throughout the pulmonary epithelium. Because they are adapted for generalized delivery to the lung, the present methods are particularly suitable in this regard.

A genetic deficiency in α₁-antitrypsin is a predisposing factor in developing familial emphysema. This protein, which normally provides much of the antiprotease protection for the lung, is produced in the liver and reaches the lung by diffusion from the serum. Thus, the use of this gene in the present methods will provide effective therapy for familial emphysema and other inflammatory conditions of the lung where the antiprotease defenses are either nonexistent or have been overwhelmed.

Pulmonary inflammatory processes are likely to be ongoing in emphysema patients. Accordingly, a vector that directly transduces the pulmonary epithelium must avoid exacerbating this condition. In addition, a suitable vector, rather than being injectable, could be aerosolized, and the promoter, without being required to be as strong as the endogenous natural liver promoter, preferably is both tissue-specific and constitutively expressed.

Though most gene therapies heretofore have concentrated on diseases with a clear genetic basis, acquired diseases of the lung and diseases with complex etiologies such as asthma are treatable by the present methods. For example, selective oxidation of anti-proteases in the smoker's lung contributes to the development of COPD, thereby altering the protease-anti-protease balance of the lung. Laurell et al., Sc. J. Clin. Lab. Invest. 15: 132-140, (1963). This balance may be restored by administering antiprotease via the present methods. Moreover, in the case of secretory leukoprotease inactivator (SLPI), a major antiprotease present in the lung, it is known that a mutation replacing methionine at position 73 of the mature protein with leucine renders the protein oxidation resistant. Stolk et al., Pulm. Pharm. 6: 33-39, (1993). Hence, treatment of COPD patients with anti-proteases, like SLPI, and especially oxidation-resistant anti-proteases, is contemplated.

Suitable SLPI proteins include the following:

SEQ ID No. 1 (native mature form):
SGKSFKAGVC PPKKSAQCLR YKKPECQSDW QCPGKKRCCP
DTCGIKCLDP VDTPNPTRRK PGKCPVTYGQ CLMLNPPNFC
EMDGQCKRDL KCCMGMCGKS CVSPVKA
SEQ ID No. 2 (native immature form):
MKSSGLFPFL VLLALGTLAP WAVEGSGKSF KAGVCPPKKS
AQCLRYKKPE CQSDWQCPGK KRCCPDTCGI KCLDPVDTPN
PTRRKPGKCP VTYGQCLMLN PPNFCEMDGQ CKRDLKCCMG
MCGKSCVSPV KA
SEQ ID No. 3 (oxidation-resistant mature form):
SGKSFKAGVC PPKKSAQCLR YKKPECQSDW QCPGKKRCCP
DTCGIKCLDP VDTPNPTRRK PGKCPVTYGQ CLLLNPPNFC
EMDGQCKRDL KCCMGMCGKS CVSPVKA
SEQ ID No. 4 (oxidation-resistant immature form):
MKSSGLFPFL VLLALGTLAP WAVEGSGKSF KAGVCPPKKS
AQCLRYKKPE CQSDWQCPGK KRCCPDTCGI KCLDPVDTPN
PTRRKPGKCP VTYGQCLLLN PPNFCEMDGQ CKRDLKCCMG
MCGKSCVSPV KA

Thus, a particularly useful class of nucleic acid is one which contains a transgene encoding a protease inhibitor. Exemplary protease inhibitors may inhibit the activity of proteases such as neutrophil elastase, cathepsin G, collagenase, proteinase 3 and plasminogen activator. The skilled artisan will recognize that inhibition of other proteases, including proteases not yet identified, can be beneficial in this regard. Particular classes of protease inhibitors inhibit serine proteases or metalloproteases, for example. Protease inhibitors useful in the inventive methods and compositions include α₁-antitrypsin, Secretory Leukocyte Protease Inhibitor (SLPI), α₁-antichymotrypsin, tissue inhibitors of metalloprotease (“TIMPs”, like TIMP-1, -2 and -3), elafin and β2-macroglobulin.

Some (α₁-antitrypsin and SLPI, for example) of the foregoing protease inhibitors are inactivated via an oxidative mechanism. In particular, they have a sulfhydryl-containing amino acid (e.g., cysteine), which must be in reduced form for maximal activity. As detailed above for SLPI, oxidation resistant analogs may be prepared that lack such residues. It is anticipated that such oxidation-resistant (and, hence, inactivation-resistant) protease inhibitors will have improved pharmacodynamic properties, such as increased half-life.

In yet another embodiment, the transgene sequence for expression can be modified to generate a protein with altered or new peptide sequences that have beneficial effects on the pharmacology of the protease inhibitor. In one aspect of this embodiment, the transgene for a protease inhibitor is modified to produce a chimeric protein with all or part of a natural or engineered immunoglobin sequence. The binding activity of the immunoglobin portion permits enhanced binding to tissue regions of interest and enhanced persistence at those tissue regions. Other peptide sequences can be used in place of the immunoglobin sequence that permit retention in the tissue or protection from metabolic processes in the tissue.

An additional, more general, approach to remediating oxidative injury to the lung, including anti-protease oxidation, is to use the present methods to deliver anti-oxidants. Anti-oxidant therapies for lung diseases employ, for example, superoxide dismutase or catalase. These antioxidant therapies are also useful generally in treating inflammatory conditions, since inflammation results in the activation of oxidative processes (e.g., myeloperoxidase), and the subject antioxidants will neutralize the resulting reactive oxygen species.

Injury to the lung also is remediable by using nucleic acids that inhibit disease processes. In one embodiment, the nucleic acid inhibits production of natural proteases. This can be through expression of antisense or ribozyme constructs or through direct inhibition of cells producing the protease.

With regard to each therapeutic gene contemplated herein, the artisan will recognize that a certain degree of variation in the primary amino acid and protein sequence is tolerable without substantially impairing the function of the underlying protein, which is the most important characteristic. Thus, the invention encompasses such variation, in the form of derivatives or variants, which terms are used interchangeably herein, and specifically include oxidation-resistant forms of protease inhibitors. In general, derivatives of both the DNA and protein molecules encompassed by the invention can be defined with reference to “sequence identity.” “Sequence identity” refers to a comparison made between two molecules using, for example, the standard Smith-Waterman algorithm that is well known in the art.

Some derivatives will have at least about 50%, 55% or 60% identity. Preferred molecules are those having at least about 65% sequence identity, more preferably at least 65% or 70% sequence identity. Other preferred molecules have at least about 80%, more preferably at least 80% or 85%, sequence identity. Particularly preferred molecules have at least about 90% sequence identity, more preferably at least 90% sequence identity. Most preferred molecules have at least about 95%, more preferably at least 95%, sequence identity. As used herein, two nucleic acid molecules or proteins are said to “share significant sequence identity” if the two contain regions which possess greater than 85% sequence (amino acid or nucleic acid) identity.

“Sequence identity” is defined herein with reference to the BLAST 2, algorithm using default parameters. See Altschul et al., J. Mol. Biol. 215:403-410 (1990); Gish, Nature Genet. 3:266-272 (1993); Madden, Meth. Enzymol. 266:131-141 (1996); Altschul et al., Nucleic Acids. Res. 25:3389-3402 (1997); and Zhang et al., Genome Res. 7:649-656 (1997). The BLAST 2 algorithm also is available at the NCBI (http://www.ncbi.nlm.nih.gov/BLAST).

Because the present methods fail to exacerbate an inflammatory condition, they may be used for treating inflammatory conditions in general, including such conditions of the lung. In contrast to known methods, the methods described herein pose little risk of contributing to the very condition sought to be treated. In the present methods, a therapeutically effective amount of transgene expression or nucleic acid activity is an amount that substantially inhibits an adverse inflammatory response.

Other embodiments of the method for treating inflammatory conditions include treatments of arthritis. In this embodiment one method is the expression of transgenes that protect the cartilage from proteases resulting from undesired inflammation in the joints. Likewise, the method can be used for delivery of nucleic acid constructs that exert an activity which diminishes one or more steps in the undesired inflammation in joints. Other embodiments of the method are for the treatment of undesired or excessive inflammation in other tissues and pathologies.

An example of an inflammatory disorder of the lungs that is treatable according to the present methods is asthma. Asthma involves a complex cascade of inflammatory mediators, any of which is a target for therapeutic intervention. Asthma is characterized predominantly by the presence of TH2-like T-cells, producing IL-4 and IL 5, but not IL-2 or IFN-γ. The C—C-chemokines, which include RANTES, MCP-3, MCP-4, Eotaxin and, Eotaxin-2. In addition, certain cytokines are known to counteract the TH2-driven inflammatory response, including IL-12, IFN-α, IFN-γ, IL-10, and TGF-β. Thus, the range of targets for the treatment of inflammatory pulmonary disorders includes, but is not limited to, IL-4, IL-5, RANTES, MCP-3, MCP-4, Eotaxin, Eotaxin-2, IL-12, IFN-α, IFN-γ, IL-10, and TGF-β.

IL-10 is considered a general immunosuppressor that inhibits IFN-γ and IL-2 production by TH2 cells, as well as a variety of other immune responses. While IL-10 is a somewhat general immune mediator, IL-5 is more specific. In particular, IL-5 is the major, and perhaps the only, cytokine involved in eosinophilia, which makes it a particularly attractive point of therapeutic intervention in eosinophilic diseases, such as allergy and asthma. Thus, preferred therapeutic targets also include IL-10 (see, e.g., GenBank Acc. Nos. U91746, U16720 and X78437 for IL-10, and 4504632 and U00672 for the IL-10 receptors) and IL-5 (see, e.g., GenBank Acc. Nos. J03478 and M33949 for IL-5, and A26251 and A26249 the IL-5 receptor).

The foregoing inflammatory mediators may be inhibited by a variety of agents that can be encoded in a gene therapy vector. For example, genes encoding specific antibodies, and especially antibody fragments may be cloned into such a vector. Where specific receptors for these mediators are involved and known, soluble forms of the receptors may be encoded in the vector. For example, two naturally-occurring forms of the IL5 receptor (“IL-5R”) exist: one is membrane bound and the other is soluble. The soluble form inhibits the binding of IL-5 to the membrane form, thereby antagonizing the biological activity of IL-5. Thus, soluble IL-5R is a preferred antiinflammatory medicament. Other inhibitors, such as antisense nucleic acids and ribozymes also may be employed. The term “treating” in its various grammatical forms as used in describing the present invention refers to preventing, curing, reversing, attenuating, alleviating, minimizing, suppressing or halting the deleterious effects of a disease state, disease progression, disease causative agent or other abnormal condition.

The foregoing discussion and following examples are not limiting of the present invention. In particular, one skilled in the art will readily recognize additional embodiments that are not specifically exemplified but that are within the scope of the invention.

### EXAMPLE 1

### Optimum Dose of Free Nucleic Acid

This example demonstrates the optimization of gene expression using free nucleic acid. The results presented below show that at all doses, gene expression was observed. However, the optimum levels of gene expression in this experiment were observed when the dose was 80 μg.

Experiments were conducted using different doses of naked DNA all diluted in 100 μl of phosphate buffered saline (PBS). As seen in Table 1, the doses used were 25, 50, 80 and 120 μg of DNA. STD is standard deviation.

TABLE 1

| Treatment | Average | STD |
|---|---|---|
| PBS | 208.75 | 39.23885 |
| 25 μg | 2949.75 | 1328.602 |
| 50 μg | 12666 | 5126.094 |
| 80 μg | 26758 | 2022.444 |
| 120 μg | 14929 | 10208.28 |

Experimental: The plasmid used was pCIluc, which contains the firefly luciferase gene under the control of the cytomegalovirus promoter. Female BALB/c mice (6-8 weeks old) were obtained from Harlan Sprague Dawley (Indianapolis, Ind.). Mice were anesthetized by placing them in a bell jar containing 5% isoflurane. The DNA was applied to the lungs by oral tracheal instillation using an angled feeding needle. Twenty four hours after administration, the animals were sacrificed, and the lungs were removed and placed on dry ice.

The luciferase activity was determined using a kit from Promega (Madison, Wis.), and a luminometer from Berthold Systems, Inc. (Pittsburgh, Pa.). Briefly, the lungs were placed in 500 μl of lysis buffer (Promega kit) and homogenized for 20 seconds with a tissue homogenizer (Brinkman Polytron). Samples were centrifuged in a microcentrifuge for 30 minutes at 14 000 g at 4° C. The protein concentrations were determined using the Bradford reagent (BioRad, NY) A sample containing 100 μg of protein from the supernatant fraction was used in the luciferase assay. Luciferase activity in each sample was normalized to the relative light units per milligram of extracted protein.

### EXAMPLE 2

### Optimizing the Composition

This example demonstrates that optimum gene expression is affected by the solution in which the free nucleic acid is diluted. The results presented below show that free nucleic acid in a 5% glucose solution yielded the highest gene expression. Furthermore, compared to a volume of 100 μl, free nucleic acid delivered in a volume of 175 μl resulted in higher levels of gene expression. The surprising finding that greater volumes of diluent resulted in higher transgene expression levels using the same mass of nucleic acid facilitated the discoveries presented in Examples 3 through 10.

The experiments were conducted using 50 μg of naked DNA per BALB/c mouse. Plasmid DNA was mixed with either H₂O, PBS or 5% glucose at two different volumes (100 and 175 μl). This mixture was administered by oral tracheal instillation into the mice. Protein concentration and luciferase assays were performed. Results are shown in Table 2.

TABLE 2

| Treatment | Average | STD |
|---|---|---|
| 100 μl H₂O | 478.75 | 84.30253 |
| 175 μl H₂O | 2761.25 | 706.1942 |
| 100 μl PBS | 3887 | 533.9819 |
| 175 μl PBS | 17273.5 | 1847.8 |
| 100 μl Glucose | 17293.75 | 2284.07 |
| 175 μl Glucose | 24481.5 | 4452.451 |

Experimental: Samples were treated essentially as described in Example 1.

### EXAMPLE 3

### Surfactant Improves Delivery of Free Nucleic Acid

This example demonstrates that surfactant-mediated vector delivery results in increased gene expression when compared to free nucleic acid.

Compositions containing 50 μg of plasmid DNA and varying amounts of surfactant were provided by oral tracheal administration. A representative surfactant, Survanta, was administered into BALB/c mice at 10, 12, 14 and 16 mg/ml. The lungs were collected at 24 hours, and protein concentration and luciferase activity were ascertained. As seen in Table 3, higher levels of expression were achieved in the presence of Survanta as opposed to control.

TABLE 3

| | | | Experiment 2 | |
|---|---|---|---|---|
| | Experiment 1 | | Average | |
| | Average | | Ex- | |
| Composition | Expression | STD | pression | STD |
| PBS | 111 | 9.128709 | | |
| DNA | 6763.25 | 790.8284 | 24495.75 | 3742.634 |
| DNA (10 mg/ml) | 8962 | 6295.523 | 41910.75 | 4558.049 |
| DNA (12 mg/ml) | 8579.25 | 4343.354 | 59728.75 | 16926.77 |
| DNA (14 mg/ml) | 12044.75 | 21841.94 | 28934 | 7381.025 |
| DNA (16 mg/ml) | 9748.75 | 2542.666 | | |

Experimental: Samples were treated essentially as described in Example 1. DNA samples were diluted in 5% glucose to a final volume of 150 μl (Experiment 1) or 175 μl (Experiment 2) and then administered to each mouse.

### EXAMPLE 4

### Comparative Gene Expression at Different Time Points

This example demonstrates the different levels of gene expression over time for compositions containing free nucleic acid and a surfactant, Survanta. The results presented in Table 4 show, the levels of gene expression were increased with the addition of surfactant after 24, 48 and 72 hours over DNA that was administered without surfactant.

The experiments were conducted by mixing 50 μg of plasmid DNA with 14 mg/ml Survanta to a final volume of 100 μl. This mixture was administered by oral tracheal instillation to BALB/c mice. Protein concentration and luciferase assays were performed. N DNA is DNA alone and DNA/Su is DNA with surfactant.

TABLE 4

| | Average | |
|---|---|---|
| Composition | Expression | STD |
| PBS | 111 | 9.128709 |
| N DNA (24 hr) | 3334.75 | 1742.172 |
| DNA/Su (24 hr) | 8159 | 6286.186 |
| DNA/Su (48 hr) | 11830.75 | 11921.67 |
| DNA/Su (72 hr) | 10496.75 | 5454.225 |

Experimental: Samples were treated essentially as described in Example 1.

### EXAMPLE 5

### Optimizing the Volume of Administration

This example demonstrates how to optimize the volume of administration. In sum, the quantity of vector and surfactant were held constant, and the volume of composition was varied.

Experiment 1 was conducted using compositions containing 50 μg of plasmid DNA, 14 mg/ml of surfactant, and the remaining volume being 5% glucose. The final volumes of the mixture were 75, 100, 120, 150 and 175 μl. Experiment 2 utilized 50 μg DNA in PBS without surfactant in volumes of 100, 125, 150, 175 and 200 μl. Protein and luciferase assays were preformed. Results are shown in Table 5.

TABLE 5

| | Experiment 1 | | Experiment 2 | |
|---|---|---|---|---|
| Final | Average | | Average | |
| Volume | Expression | STD | Expression | STD |
| 75 μl | 684 | 164.8257 | | |
| 100 μl | 3911.5 | 1928.204 | 7296.75 | 1582.134 |
| 120 μl | 4930.333 | 593.1546 | | |
| 125 μl | | | 9817.5 | 1662.179 |
| 150 μl | 11305.25 | 9313.851 | 20297.75 | 11546.76 |
| 175 μl | 4497.75 | 1737.65 | 43576.75 | 11354.37 |
| 200 μl | | | 36597.38 | 4269.023 |
| 100 μl H₂0 | | | 4296.75 | 1964.947 |

Experimental: Samples were treated essentially as described in Example 1.

### EXAMPLE 6

### Duration of Gene Expression Upon a Single Oral Tracheal Administration of Free Nucleic Acid

This example demonstrates that, after a certain period of time, gene expression reached a peak, whereafter the gene expression gradually declined until it finally reached a baseline level. The results presented below show that the gene expression started after 6 hours. The gene expression clearly started declining after 24 hours and reached its baseline by day 6.

Experiments were conducted using 80 μg of naked DNA in a volume of 100 μl of PBS. As seen in Table 7, BALB/c mice were harvested at time points 6, 12, 24, 48 and 72 hours and 6, 10, 14, 21 and 28 days.

TABLE 6

| | Average | |
|---|---|---|
| Treatment | Activity | STD |
| 6 hrs | 11114.4 | 3682.37 |
| 12 hrs | 9503 | 1998.875 |
| 24 hrs | 9968.2 | 1123.176 |
| 48 hrs | 6629.4 | 2385.795 |
| 72 hrs | 6987 | 899.3717 |
| 6 days | 3111.4 | 619.9881 |
| 10 days | 331 | 109.5084 |
| 14 days | 448.6 | 277.1395 |
| 21 days | 146.6 | 22.08619 |
| 28 days | 191 | 67.21235 |
| PBS | 208.8 | 39.32302 |

Experimental: Samples were treated essentially as described in Example 1.

### EXAMPLE 7

### Repeated Delivery of Free Nucleic Acid Surprisingly Results in Expression Levels that can be Repeatedly Achieved, with No Limiting Inflammatory or Immune Response

This example demonstrates that the present methods can be performed repeatedly, such that expression of the delivered transgene can be repeatedly achieved. The results presented below show that when 50 μg of plasmid DNA was readministered every seven days for 28 days, gene expression was sustained to levels seen after the original plasmid DNA administration.

This experiment was conducted by repeatedly administering 50 μg of naked DNA in 150 μ□l of 5% glucose on days 0, 7, 14, 21 and 28. Protein concentration and luciferase assays were performed on lung homogenates harvested 24 hours following a DNA administration. Measurements were taken at the indicated times following all previous doses (post) or with all but the dose administered 24 hours previously (pre). Results are shown in Table 7. Thus, in this example, expression levels peaked shortly after each DNA dose then gradually declined following kinetics similar to those observed with a single DNA dose, as seen in Example 6.

TABLE 7

| Day | Average | STD |
|---|---|---|
| 1 (pre) | 142 | 5.522681 |
| 1 (post) | 10611.6 | 2044.122 |
| 8 (pre) | 523.4 | 285.3722 |
| 8 (post) | 6976.8 | 1828.562 |
| 15 (pre) | 247.6 | 49.92795 |
| 15 (post) | 13528.4 | 6640.327 |
| 22 (pre) | 792.2 | 70.50674 |
| 22 (post) | 7604.6 | 1158.241 |
| 29 (pre) | 634.4 | 201.2965 |
| 29 (post) | 12465.75 | 4197.746 |

Experimental: Samples were treated essentially as described in Example 1.

### EXAMPLE 8

### Repeated Delivery of Free Nucleic Acid does not Result in Persistent Expression in Mice when Administered Every 48 Hours

Although repeated delivery of free nucleic acid in a one week period lead to repeated gene expression, this example demonstrates that persistent expression is not achieved in mice when the readministration is every 48 hours for about at least a week. While readministration after the initial 48 hour period lead to a sizeable increase in gene expression, subsequent administrations failed to evoke further gene expression.

Experiments were conducted using 80 μg of plasmid DNA in a 5% glucose solution to a final volume of 175 μl. The mixture was administered on days 3, 5, 7, 9, 11, 13 and 15. Protein concentration and luciferase assays were performed. Results are shown in Table 8.

TABLE 8

| | Average | |
|---|---|---|
| Day | Activity | STD |
| Day 3 | 17190.80 | 1064.4 |
| Day 5 | 31161.20 | 6703.619 |
| Day 7 | 5976.40 | 2608.461 |
| Day 9 | 273.00 | 216.3631 |
| Day 11 | 308.40 | 230.1028 |
| Day 13 | 343.00 | 102.7643 |
| Day 15 | 276.20 | 38.41484 |

Experimental: Samples were treated essentially as described in Example 1, except that five mice were treated every 48 hours.

### EXAMPLE 9

### Using a Supplemental Antiinflammatory Regimen

This example demonstrates that the administration of an antiinflammatory compound in conjunction with the above-described protocol allows for more frequent vector administration, which results in greater overall sustained and persistent levels of transgene expression.

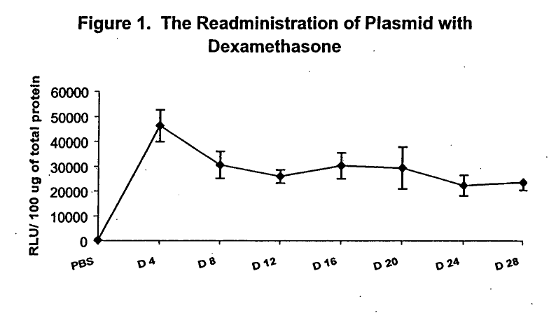

Experimental: This experiment was conducted using 80 μg of pCIluc plasmid in a 5% glucose solution to a final volume of 160 μl. The mixture was administered by oral-tracheal delivery on days 0, 4, 8, 12, 16, 20 and 24. On days −1, 0 and 1, the mice received 10 mg/kg dexamethasone, intraperitoneally. Additionally, the mice received 10 mg/kg dexamethosone (ip) thirty minutes prior to plasmid delivery on days 4, 8, 12, 16, 20 and 24. Animals were sacrificed 4 days following plasmid administration and protein concentration and luciferase assays were performed. Results are presented below in FIG. 1 and are reported as mean+/−standard deviation. Data points represent five animals each, except day 24, which utilized only four animals.

### EXAMPLE 10

### Tissue Distribution of a Secreted Transgene Product

This example demonstrates the distribution of a protease inhibitor transgene product, following oral-tracheal instillation of a plasmid, versus intravenous administration and administration with purified protein. These data show that the transgene delivers similar amounts of product as direct delivery of the protein. Because the protein is constantly made by the transgene, overall levels do not decrease over time as is the case with the protein, providing a clear benefit of the present methods. Data were collected twelve hours after oral-tracheal or intravenous administration.

Experimental: These experiments employed the vector pCIhSPLI, which contains the human cDNA sequence of secretory leukoprotease inhibitor (SLPI) driven by the CMV promoter. Stetler, et al., Nucleic Acids Res. 14: 7883-7896 (1986). Oral tracheal instillations (OT) were performed on a with 80 μg of pCIhSLPI diluted in 5% glucose to a final volume of either 150 μl. Additionally, a group of BALB/c mice was instilled via the OT route with 1 μg recombinant hSLPI protein in 150 μl 5% glucose (HSLPI-protein).

DOTAP (1,2-dioleoyoxy-3-(trimethylammonio)propane)-cholesterol complexed pCIhSLPI was prepared for intravenous (IV) injection. Forty five mg DOTAP and 25 mg cholesterol were mixed in cyclohexane and lyophilized to dryness. Double distilled water was added to the lipid cake to give a final concentration of 10 mg/ml of cationic lipid (i.e., not including the cholesterol component) and allowed to hydrate at 70° C. for 1 hour. The DOTAP/cholesterol dispersion was extruded through 100 nm pore carbonate membranes (Avanti Polar Lipids, Inc.). The resulting size of the DOTAP-cholesterol dispersion was 150-200 nm. Intravenous (IV) injections (200 μl) were performed using 60 μg pCIhSLPI complexed with DOTAP-cholesterol at a 4 to 1 (positive to negative) charge ratio. The lipoplexes were prepared immediately prior to IV injection by adding the 60 μg of plasmid, dissolved in 100 μl 10% glucose, to 100 μl of 5.1 mg/mil DOTAP-cholesterol dispersion in ddH₂O.

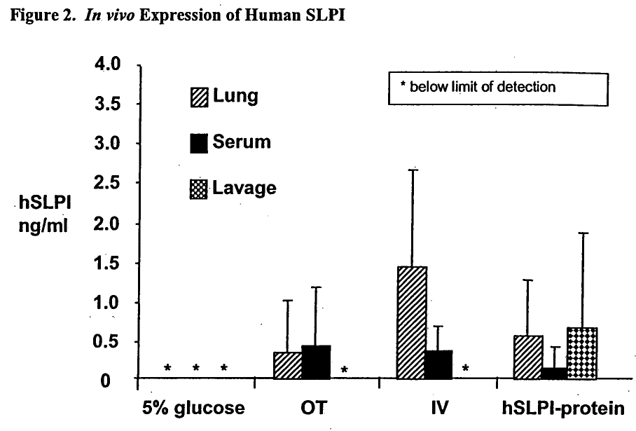

Twelve hours following administrations, the animals were sacrificed and bronchoalveolar lavage (BAL) was performed by annulating the trachea with a silastic catheter and slowly injecting 700 μl of PBS. Lungs were collected and placed with 500 μl lysis buffer in lysing matrix tubes (BIO 101, Vista, Calif.). The lungs were homogenized for 20 seconds at speed 6.0 in a Fast Prep FP120 (BIO 101). The homogenate was microcentrifuged at 14 000 g at 4° C. for 30 minutes. Additionally, blood was collected, allowed to clot and the sera were separated. The recovered fluids were then centrifuged at 1500 rpm for 10 minutes. The supernatant fractions were removed and assayed for the presence of human SLPI using a double-antibody sandwich enzyme-linked immunosorbent assay (ELISA) kit (R&D Systems, Minneapolis, Minn.) following the recommended protocol. Data are reported as means+/−standard deviations (FIG. 2).

We claim:

1. A non-inflammatory vector composition, comprising (i) aqueous free nucleic acid that encodes a gene product and (ii) an antiinflammatory compound.

2. A composition according to claim 1, further comprising (iii) an enhancing agent.

3. A composition according to claim 2, wherein said enhancing agent is a surfactant.

4. A composition according to claim 3, wherein said surfactant is selected from the group consisting of Survanta, Exosurf, Infasurf, Pluronic, anionic liposome formulations, Thesit, Brij 58, Brij 78, Tween 80, and Chol-PEG 900.

5. A composition according to claim 2, wherein said enhancing agent is a polysaccharide.

6. A composition according to claim 5, wherein said polysaccharide is a linear repeating disaccharide unit of 1,4-linked β-D-glucuronic acid and 1,3-linked 2-acetamido-2-deoxy-β-D-glucopyranose.

7. A composition according to claim 1, wherein said antiinflammatory agent is a steroid.

8. A composition according to claim 7, wherein said steroid is selected from the group consisting of beclomethasone, triamcinolone, flunisolide, fluticasone, budesonide dexamethasone and hydrocortisone.

9. A composition according to claim 1, wherein said gene product is a protease inhibitor.

10. A composition according to claim 9, wherein said protease inhibitor inhibits the activity of a protease selected from the group consisting of neutrophil elastase, cathepsin G, collagenase, gelatinase, proteinase 3, and plasminogen activator.

11. A composition according to claim 10, wherein said protease inhibitor is selected from the group consisting of α1-antitrypsin, Secretory Leukocyte Protease Inhibitor, α1-antichymotrypsin, TIMP-1, elafin, β2-macroglobulin and derivatives thereof.

12. A composition according to claim 11, wherein said protease inhibitor is Secretory Leukocyte Protease Inhibitor or an oxidation-resistant form thereof.

13. A method of treating a patient suffering from a disorder having an inflammatory component, comprising at least twice administering to the patient an effective amount of a non-inflammatory vector composition, comprising (i) aqueous free nucleic acid that encodes a gene product and (ii) an enhancing agent.

14. A method according to claim 13, wherein said administration is tracheal or intra-articular.

15. A method according to claim 14, wherein said administration is by aerosolization or by intra-articular injection.

16. A method according to claim 13, wherein administration is tracheal and further comprises administering an antiinflammatory agent by intravenous or oral route, prior to administering said composition.

17. A method according to claim 13, wherein said disorder is associated with pulmonary or intra-articular inflammation.

18. A method according to claim 17, wherein said disorder is selected from the group consisting of emphysema, chronic obstructive pulmonary disease (COPD), cystic fibrosis (CF), adult respiratory distress syndrome (ARDS) and asthma.

19. A method according to claim 17, wherein said disorder is selected from the group consisting of rheumatoid arthritis and osteoarthritis.

20. A method of treating a patient suffering from a disorder having an inflammatory component, comprising at least twice administering to the patient an effective amount of a non-inflammatory vector composition, comprising (i) aqueous free nucleic acid that encodes a gene product and (ii) an immunosuppressive agent.
